(19) United States
(12) Patent Application Publication
(10) Pub. No.: US 2014/0364683 A1
(43) Pub. Date: Dec. 11, 2014

(54) ILLUMINATION SIGNAL, SYSTEM AND METHOD

(71) Applicant: KONINKLIJKE PHILIPS N.V., EINDHOVEN (NL)

(72) Inventors: Gary Nelson Garcia Molina, Madison, WI (US); Giovanna Wagenaar Cacciola, Best (NL); Mirela Alina Weffers-Albu, Boukoul (NL); Tsvetomira Kirova Tsoneva, Eindhoven (NL)

(73) Assignee: KONINKLIJKE PHILIPS N.V., EINDHOVEN (NL)

(21) Appl. No.: 14/363,564

(22) PCT Filed: Nov. 23, 2012
(86) PCT No.: PCT/IB2012/056652

§ 371 Date: Jun. 6, 2014

Related U.S. Application Data
(60) Provisional application No. 61/567,705, filed on Dec. 7, 2011.

(51) Int. Cl. A61M 21/02

(52) U.S. Cl. CPC A61M 21/02; A61M 2021/0044
USPC 600/27

(57) ABSTRACT

An illumination system is provided comprising a light source and a controller and being configured to provide an illumination signal (15) for, when perceived by a mammalian, in particular human, subject, inducing relaxing in the subject. The signal comprises a plurality of light pulses (17) having a pulse duration (T17) and being separated by inter-pulse intervals (19). The light pulses are grouped in stimuli (21) which have a stimulus duration (T21) and are separated by inter-stimuli intervals (23). The stimuli are grouped in stimulation sequences (25) which have a stimulation sequence duration (T25) and are separated by inter-sequence intervals (27). An illumination signal, a method, and a computer program product are also provided.

### FIELD OF THE INVENTION

The present disclosure relates to an illumination system comprising a light source and a controller and being configured to provide an illumination signal for, when perceived by a mammalian, in particular human subject, inducing relaxing in the subject. It further relates to an illumination signal, a method, and a computer program product.

### BACKGROUND OF THE INVENTION

In many circumstances persons may experience stress, be excited and/or be normally awake whereas a relaxed state is desired by the person or beneficial to the person. Examples of such circumstances are waiting for a potentially stressful experience, e.g. in a doctors' or dentists' waiting room, recovering from illness or surgery, or generally desiring to relax and/or sleep.

It is well known that light levels can influence mammalian, in particular human behaviour, e.g. in adjustment of the circadian rhythm. E.g., US 2010/0130812 discloses light modulation devices. At least one Low Frequency Oscillator is used to create an oscillating signal used to drive an intensity parameter, a color parameter or both in a light modulator. The oscillating signal may be mixed with a base signal. The modulated signal driving either of the intensity or color parameters may be simple or complex. Systems having a plurality of light projection devices, each associated with a corresponding light modulation device, are also provided. Such light modulation systems may be used for a variety of applications. In particular, US 2010/0130812 teaches the use of gradual modulation of light intensity or color in frequency ranges corresponding to brain wave frequencies, in order to reduce stress or induce relaxation in a person.

Further improvements are, however, desired.

### SUMMARY OF THE INVENTION

Herewith, an illumination system according to claim 1 is provided. It has been found that when the illumination signal is perceived by a person, a sense of relaxation is induced in the person. It is believed that the same holds for other mammalian subjects than humans. The system enables administration of the signal to a subject to induce such relaxation.

The origin of the relaxation sensation is attributed to alternating periods of increased arousal or increased brain activity in general, excited by perceiving stimuli of series of rapid sensory impulse variations, and periods of relaxation when realizing that the stimulus is absent, wherein in successive occurrences of inter-stimuli intervals the levels of relaxation tend to deepen and the levels of brain activity or arousal caused by successive stimuli tend to decrease. The result is a trend towards deeper relaxation. This trend is amplified by a similar process on a different time scale by the grouping of stimuli in stimulation sequences which are likewise separated by inter-sequence intervals.

Millisecond pulses are generally detectable by humans. Intermittent light signals in a range of about 40-100 Hz are resolved by the human visual system (eye and brain) and provoke arousal, e.g. see M. Pastor, et al., “Human cerebral activation during steady-state visual-evoked responses,” The Journal of Neuroscience, 23(37): 11621-7, 2003. Although signals having a pulse repetition rate of over 60 Hz may consciously be perceived as continuous, they still generally provide a neural response in the brain indicative of detection as a series of individual signals instead of constant signal levels, and provoke the desired initial brain activity e.g. see A. K. Probadnigk, et al, “Revealing the Neural Response to Imperceptible Peripheral Flicker with Machine Learning”, Conf Proc IEEE Eng Med Biol Soc, 2011: 3692-5, 2011.

A pulse repetition rate of 40-60 Hz is preferred since this is consciously detectable as flickering, thus, the subject can be clearly aware of the signal. This may increase acceptance of a sensation of arousal by the subject and lower the degree of arousal and/or it may reduce chances of inadvertent anxiety. Thus, effectiveness of the relaxation induction may be increased.

Short series of pulses may already be detected, at least sub-consciously, but they may be mentally treated as “noise” and thus be neglected. It has been found that a stimulus duration of about 300 milliseconds (ms) is generally sufficient to ensure detection of the stimulus as a “signal” and to cause the desired neural response of arousal. A longer stimulus duration increases the subjects' awareness of the stimulus and therewith its impact and acceptance. Extended stimulus durations, typically of more than several tens of seconds, e.g. about 30 s, have limited added value and may even cause an effect of annoyance, which would reduce the relaxation effect and should preferably be avoided.

A stimulus duration in the range of about 1-10 seconds is therefore preferred, e.g. in a range of about 2-5 s. It is noted that annoyance may also be avoided by increasing the pulse repetition rate to above conscious detection, e.g. above about 60 Hz.

For improved effect, subsequent stimuli should be clearly detected as separate entities and not as a continuation of each other. This may be achieved with inter-stimuli intervals being significantly longer than the inter-pulse intervals within each stimulus. It has been found that inter-stimuli intervals of a few seconds generally suffice, e.g. 2 seconds, preferably at least 3 seconds. Longer intervals, up to a few tens of seconds may be employed to increase mental separation of individual stimuli. Even longer intervals, e.g. more than about 30 seconds, may reduce the effect of the signal, since at each new stimulus the effects of the previous stimulus and its absence may have decayed so far that, as it were, the previous stimulus and its effects have been “forgotten”. Inter-stimuli intervals in the range of about 2-30 seconds are therefore preferred, more preferably they are on the order of about 10 seconds or shorter e.g. in a range of about 8-12 s or about 3-6 s.

Similar to the stimuli, the individual sequences of stimuli should preferably be clearly identifiable. Stimulation sequence durations on the order of tens of seconds have proven effective. Extended durations of over several minutes may lead to decay of the efficiency and/or even annoyance. Hence, it is preferred that at least some stimulation sequence durations are in a range of about 30 seconds to about 5 minutes. Sequence durations of about 45-90 seconds have proven particularly effective.

The length of inter-sequence intervals may be shorter than the sequences themselves, but sufficiently long to facilitate discerning sequences of stimuli from individual stimuli. Inter-sequence intervals may therefore be in the order of 5 seconds to about 45 seconds. A range of about 5-20 seconds has been found effective, with preferred ranges of about 8-12 s and in a range of about 13-16 s.

The length of the signal may have a duration of a predetermined number of sequences and/or a predetermined duration. A duration of about 10 minutes tends to suffice for achieving relaxation. Longer signals may provide deeper relaxation. A duration of over 45 minutes appears to have limited added value, shorter than 45 minutes, e.g. 30 minutes or shorter is therefore preferred. A signal duration of about 15-25 minutes, e.g. about 20 minutes, appears reliably effective and sufficiently of acceptable duration to be experienced as a short and predictable duration thereby to prevent annoyance boredom and/or fatigue so that such duration may be preferred in particular.

The pulse duration and the inter-pulse intervals may be arranged to provide a particular pulse duty cycle, wherein the duty cycle is determined by Duty Cycle=(pulse duration)/(pulse duration+inter-pulse interval), or: the fraction “on” time of a pulse compared to a cycle of a pulse and a subsequent inter-pulse interval. The duty cycle may be in a range of about 0.2-0.8, preferably in a range 0.5-0.8, so that predominantly light is on. This provides a good effect with little chance of annoyance.

The illumination system enables administration of the signal to a subject to induce relaxation of the subject.

To improve detection of the signal by the subject, the device may be configured to provide at least some light pulses with an effective light level modulation depth of about 0.2 or more, or an effective light level modulation of about 50 lux or more. Light level modulation is the level difference provided by a pulse: level modulation=(Level at pulse on−Level at pulse off). Effective light level modulation is the light level modulation over a background light level. Modulation depth represents the effective level modulation relative to the level provided by the pulse, or the difference of a pulse over the background relative to the level provided by the pulse, resulting in a number between 1 and 0: modulation depth=(Level at pulse on−Level at pulse off)/(Level at pulse on+Level at pulse off).

Preferably the modulation depth is more than about 0.3 and/or the light level modulation is more than about 200 lux, e.g. 300 lux, over ambient light. Since ambient light in an artificially lit room is generally about 200-300 lux, the light level during a pulse may be increased to a value in a range of about 400-500 lux, although higher values may be used. However, modifying a background illumination level of about 200 lux with a modulation depth of about 0.25 corresponding to a light level modulation of about 50 lux may be sufficient.

Although particular light colors or colour ranges may suitably be employed, it has been found that light having a limited spectrum in the range of about 700 to 600 nanometers (red to orange) or white light with a colour temperature of about 4000 Kelvin or more is very effective.

It is noted that in this text, “light” means electromagnetic radiation with a wavelength of about 800 nanometer to about 400 nanometer, generally encompassing the visual spectrum.

The signal may be provided superposed on another signal configured to influence behaviour of the subject. In a particular embodiment, the system may therefore comprise at least one further light source providing a further illumination signal to the subject configured to influence the subjects' behaviour, in particular the melatonin and/or cortisol cycle of the subject. Such system is particularly beneficial for treating recovering patients and/or subjects suffering from jet-lag or seasonal affective disorder.

The system may be implemented in a room illumination system, e.g. for illuminating at least part of a room with the signal.

The controller may be programmable, e.g. to provide different variants of the signal such as with different durations of pulses, stimuli, sequences, the signal and/or one or more of the respective intervals. The device may comprise a user interface for interaction with and/or programming of the controller e.g. for adaptation of the signal. A program may comprise a predetermined modification function for adjustment of the light source in dependence of input user data. Such program may be used for customization and/or personalization of the system for use with different subjects. A program may be provided by hand via the user interface and/or it may be obtained from a data storage medium e.g. a CD, DVD, memory card, USB-stick, internal memory of the appliance, etc.

A program and/or real-time operation control may also be provided via a connection and download option connected to a remote data storage medium and/or to the Internet. Real-time operation control via the Internet allows controlling and/or administering the light signal by a remote person, e.g. a therapist.

The system may suitably comprise a memory for storing one or more settings, functions, programs, etc. The system may comprise an output for putting out data stored in the memory, e.g. on a data storage medium etc. This allows portability of such data to another system and/or sharing such data with other people.

In line with the above, a signal is provided according to claim 11 and a method is provided according to claim 12. The signal may be used to the advantage to induce relaxation in a mammalian, in particular human, subject perceiving the signal. With the method, the signal may be provided.

To induce relaxation of a subject, the method may comprise illuminating the subject with light from a light source, according to the illumination signal.

The method may further comprise providing a further illumination signal to the subject configured to influence the subjects' behaviour, e.g. for influencing the circadian rhythm of the subject and/or treating seasonal affective disorder.

It is noted that during the signal, the pulse durations, stimulus durations, sequence durations and/or the respective intervals need not be constant, but may vary. Such variations may comprise increasing and/or decreasing of one or more of these parameters according to one or more deterministic and/or random patterns. Further, also or in addition to variations in timing of the signal, the signal strength in terms of light level, duty cycle, modulation depth and/or colour temperatures may be varied according to one or more deterministic and/or random patterns.

### BRIEF DESCRIPTION OF THE DRAWINGS

The above-described aspects will hereafter be more explained with further details and benefits with reference to the drawings showing an embodiment of the invention by way of example.

FIG. 1 shows an illumination system illuminating a person in a bed with an illumination signal as provided herein;

FIG. 2 indicates a build-up of the illumination signal;

FIGS. 3 and 4 show results of an exemplary use in control experiments of a system and method as provided herein.

### DETAILED DESCRIPTION OF EMBODIMENTS

It is noted that in the drawings, like features may be identified with like reference signs. It is further noted that the drawings are schematic, not necessarily to scale and that details that are not required for understanding the present invention may have been omitted. The terms “upward”, “downward”, “below”, “above”, and the like relate to the embodiments as oriented in the drawings. Further, elements that are at least substantially identical or that perform an at least substantially identical function are denoted by the same numeral.

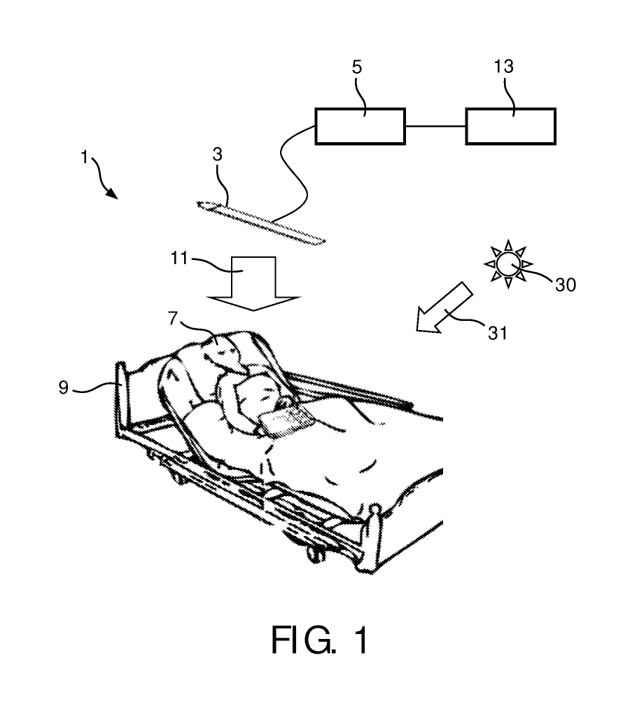

FIG. 1 shows an illumination system 1 comprising a light source 3 and a controller 5, configured to illuminate a person 7, here reclining on a bed 9, with light 11 from the light source 3. The controller 5 is coupled with an optional memory 13 in which a program may be stored for execution by the controller 5. The controller 5 is configured to operate the light source 3 to provide an illumination signal, e.g. when executing a program in the memory 13. The light source 3 may comprise one or more light emitting diodes (LEDs) which enable significant output powers and which also allow fast switching and accurate control over the emitted light power, colour and/or colour temperature. The system 1 may comprise plural light sources 3 coupled with the controller. In the shown embodiment the light source 3 comprises a plurality of LEDs arranged in an elongated shape. However, other suitably controllable light sources may be provided. The system 1 here also comprises a further light source 30 providing a further illumination signal 31 to the subject configured to influence the subjects' 7 behaviour, in particular influencing the subjects' circadian rhythm.

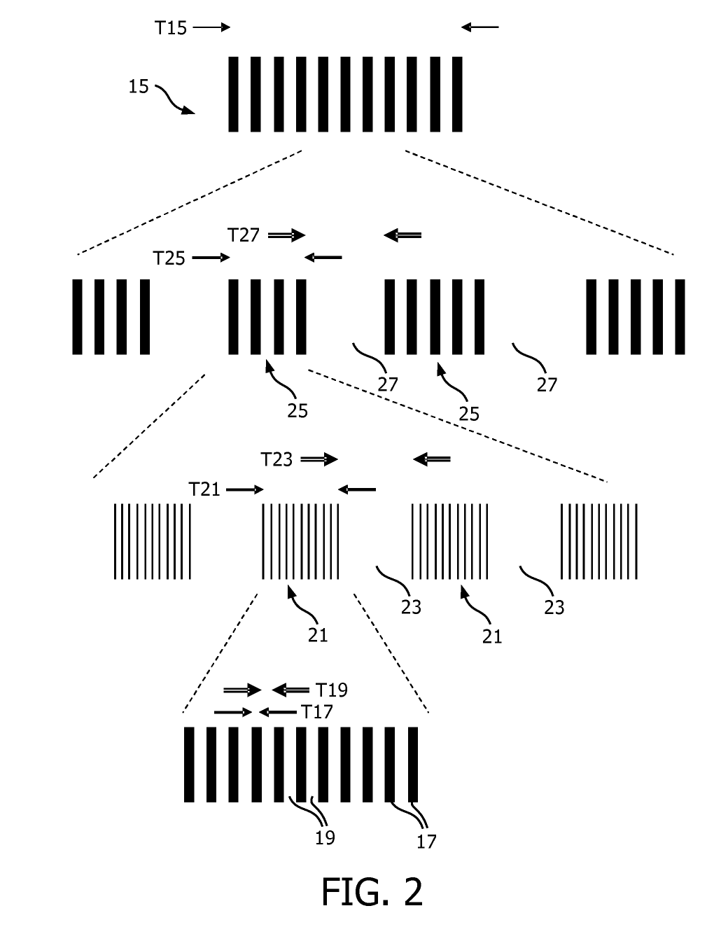

The illumination signal 15 delivers oscillatory light stimulation. A graphical representation of the timing of the signal 15 is shown in FIG. 2, wherein each row depicts a detail on successively larger scale as indicated by the broken lines. FIG. 2 indicates that the signal 15 comprises (from bottom to top) a plurality of successive light pulses 17 having a pulse duration T17 and being separated by inter-pulse intervals 19 with an inter-pulse interval duration T19. The light pulses 17 are grouped in stimuli 21 which have a stimulus duration T21 and are separated by inter-stimuli intervals 23 with an inter-stimuli interval duration T23. The stimuli 21 are grouped in stimulation sequences 25 which have a stimulation sequence duration T25 and are separated by inter-sequence intervals 27 with an inter-sequence interval duration T27.

The light pulse duration T17 and inter-pulse interval durations T19 are in the order of milliseconds to tens of milliseconds, the stimulus durations T21 are on the order of hundreds of milliseconds to tens of seconds, the inter-stimuli intervals 23 last on the order of seconds to tens of seconds, the stimulation sequence durations T25 are on the order of tens of seconds to minutes, the inter-sequence intervals 27 last on the order of seconds to tens of seconds, being longer than the inter-stimuli intervals.

Thus, the light stimulation signal 15 comprises a series of sequences 25 of light stimuli 21 separated by inter-stimuli intervals 23 which may be random. During the light stimuli 21, which may last for few seconds only (e.g. 2 seconds in a validation experiment, see below), light pulses 17 provide an oscillatory light 11, rendered by pulsing the LEDs of the light source 3. The frequency of the stimulation pulses 17 may typically be in a range from 40 to 60 Hz. The inter-stimuli intervals 27, wherein no light stimulation is rendered typically last for about ten seconds. The total duration of each such sequence 25 is of about one minute.

The rationale behind this type of visual stimulation relies on studies on the influence of light on brain activity, in particular, the brain activity entrainment that occurs when oscillatory visual stimulation is presented to a subject. By modulating the brain activity through illumination with the light signal 15, behavior is influenced (see also below). The application of a series of short light stimuli 21 with relatively long breaks in between the stimuli provided by the inter-stimuli intervals 23 decrease the arousal level in the subject and provide relaxation, in some cases even leading to sleepiness. The effect is attributed to result from the influence of the stimuli 21 on a cognitive fatigue mechanism. Randomness in the signal 15 may accelerate fatigue and thus accelerate and/or deepen relaxation.

As an example, an experiment was conducted. Five persons (S1 to S5) participated in this experiment. They were requested to sit in front of a light source 3 positioned approximately one meter away from their eyes. The light source 3 consisted of twelve LEDs, arranged in a 3×4 configuration, which shone through a white diffusion screen. The color temperature of this light source was 4441 K and the light luminance was 460 Cd/m².

The participants were exposed to a signal 15 of ten visual stimulation sequences 25. Each sequence consisted of ten 2-second-long light stimulation periods, e.g. stimuli 21 with a duration T21 of 2 seconds, and being separated with intervals (cf. inter-stimuli intervals 23) having random interval durations of 3 to 6 seconds. Between two consecutive stimulation sequences (cf. sequences 25) there was a break (cf. inter-sequence break 27) lasting about 13 to 16 seconds. Each stimulation session lasted for about 13 minutes.

Each subject participated in three stimulation sessions. In each session a different condition for the illumination was tested. The conditions were:

A: Oscillatory light stimulation. In this session, each subject was exposed to a signal 15 according to FIG. 2. In each of the ten visual stimulation sequences 25, a repetition frequency of the light pulses 17 (including inter-pulse intervals 19) was first randomly selected from the set F={40, 42, 44, 46, 48 52, 54, 56, 58, 60 Hz} and the corresponding light stimulation periods consisted of a stimulus 21 of the light source flickering at the selected frequency. The random frequency selection was such that all the frequencies in the set F were used in the signal 15.

B: Noise. In this session, in each light stimulation period (corresponding to a stimulus 21), the light intensity was varied in a random order. All the LEDs in the array were driven by Gaussian noise. The changes in intensity could not be perceived. Since noise is considered not to influence brain activity, this session was used as a control condition.

C: Lights switched off (no-light). Since no light stimulation period was provided, this session condition was used for further comparison.

For each subject, the three sessions (A—signal 15, B—noise and C—no-light) occurred at the same time on different days to avoid the influence of circadian variations. The lighting conditions in the room, the position and the duration of the experiment were kept constant across sessions. The participants were instructed to sit calmly, restrain themselves from movement and attend to the light source 3 irrespective of it emitting light or not.

At the beginning (before stimulation) and at the end (after stimulation) of each session, the subjects were asked to fill in a questionnaire measuring subjective perception of stress and relaxation, according to the Stress Adjective Check List (SACL) from C. Mackay, et al., “An inventory for the measurement of self-reported stress and arousal,” The British journal of social and clinical psychology, 17(3): 283-4, 1978.

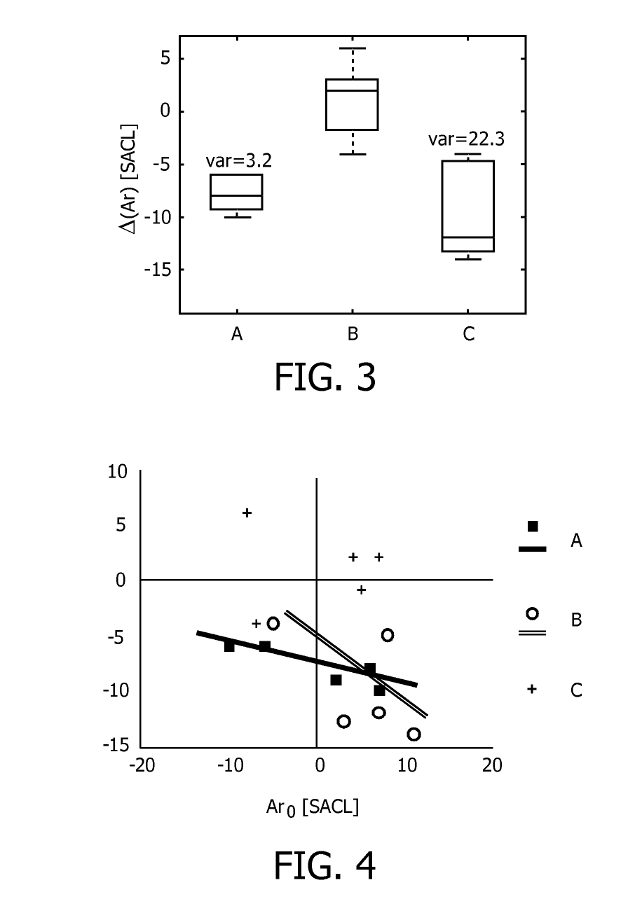

FIG. 3 shows the difference Δ (Ar) between the arousal levels, as measured by the SACL, after and before exposure to the signal 15, showing the spread of Δ (Ar) across subjects for the three conditions of oscillatory light according to the signal 15 (A), noise (B), and no-light (C) with the respective statistical variances indicated. FIG. 4 shows the data in a scatter plot with the difference Δ (Ar) vs. the initial level of arousal Ar₀ (before stimulation) with linear fits to the data (squares and solid line: signal 15 (A); circles and open line: noise (B); crosses and no line: no-light (C)).

FIGS. 3 and 4 show that the noise condition (C) did not change the arousal level. This is an expected result because in the noise condition the changes in the light intensity could not be perceived, and/or were neglected by the brain. Both in case of the oscillatory light (B) according to the signal 15 and no-light conditions (C) the arousal level was reduced. However, it is clear that the variance of the oscillatory light (B) is significantly smaller than that of the no-light condition (C). For certain subjects, sitting calmly results in relaxation, but for other subjects this has no effect, leading to a very low correlation of 0.32. In marked contrast, exposure to the oscillating light 11 with the signal 15 from the system 1 caused a reduction of the arousal level in the subjects by an almost constant value of 7 points in the SACL scale, with a high correlation coefficient of about 0.79.

The device 1 and/or the signal 15 may be adapted, e.g. be personalized, to the subject to be treated, e.g. by suitable programming of the controller 5. E.g., the light output of the light source 3 and/or the signal 15 may be adapted to the capability of the subjects' eye to adapt to a changing light intensity, e.g. as a result of the age of the subject since both pupil size and achievable variation of pupil size tend to decrease significantly with age. The adaptation may comprise increasing the number of light pulses 17 per stimulus 21 and extending the stimulus duration T21, and/or increasing the light level per pulse 17, but increasing the duty cycle of the light pulses 17 and/or adaptation of the pulse repetition rate may also be used.

The system can be applied in a healing patient room in order to improve healing and reduce length of stay of the patients in the hospital. The possibility to adapt to patients of different age groups makes it suitable for rehabilitation, children's or seniors' (geriatric) hospitals as well as elderly care facilities. The system can also be applied in waiting and intake rooms, where patients are generally under stress of an upcoming diagnose and/or a test result.

The system 1 and method to assist relaxation may also be used at home. E.g., to help people to easily fall asleep. The system 1 may be provided as a stand-alone system, e.g. incorporated in a dedicated luminaire, and it may be incorporated in existing illumination systems. E.g., a method of modification of an existing illumination system comprising a light source may comprise providing the existing system with a controller 5 that is coupled with the light source and configured to control the light source so as to provide the illumination signal 15. The method of modification may comprise providing the existing system with one or more additional light sources 3 to emit the light 11 according to the signal 15.

The system, in particular the light source 3, may be battery operated. It is noted that the system may comprise a human wearable device, e.g. a head gear, or a portable device, e.g. a torch-like device or a hand-held appliance such as a mobile telecommunications device, facilitating use without affecting others, private use and/or self-administration of the signal 15 at various locations, e.g. when travelling, where its use may assist resetting the body clock for sleeping after jet-lag.

With some modifications, one skilled in the art may extend the embodiments described herein to other architectures, networks, or technologies.

One embodiment of the disclosure may be implemented as a program product for use with a computer system. The program(s) of the program product define functions of the embodiments (including the methods described herein) and can be contained on a variety of computer-readable storage media. The computer-readable storage media can be a non-transitory storage medium. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., floppy disks within a diskette drive or hard-disk drive or any type of solid-state random-access semiconductor memory, flash memory) on which alterable information is stored.

It is to be understood that any feature described in relation to any one embodiment may be used alone, or in combination with other features described, and may also be used in combination with one or more features of any other of the embodiments, or any combination of any other of the embodiments. Moreover, the invention is not limited to the embodiments described above, which may be varied within the scope of the accompanying claims.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An illumination system comprising a light source and a controller and being configured to provide an illumination signal for, when perceived by a mammalian, in particular human, subject, inducing relaxing in the subject,
wherein the signal comprises a plurality of light pulses having a pulse duration and being separated by inter-pulse intervals, and
wherein the light pulse durations are on the order of milliseconds to tens of milliseconds and the inter-pulse intervals last on the order of milliseconds to tens of milliseconds, wherein the light pulses are grouped in stimuli which have a stimulus duration and are separated by inter stimuli intervals and the stimuli are grouped in stimulation sequences which have a stimulation sequence duration and are separated by inter-sequence intervals wherein
the stimulus durations are on the order of hundreds of milliseconds to tens of seconds,
the inter-stimuli intervals last in the order of seconds to tens of seconds,
the stimulation sequence durations are on the order of tens of seconds to minutes, and the inter-sequence intervals last in the order of seconds to tens of seconds and are longer than the inter-stimuli intervals.

2. The illumination system of claim 1, wherein the pulse duration and the inter-pulse intervals are arranged to provide a pulse repetition rate in a range of about 40-100 Hz.

3. The illumination system of claim 1, wherein at least some stimuli have a stimulus duration in a range of about 300 milliseconds to about 30 seconds.

4. The illumination system of claim 1, wherein at least some inter-stimuli intervals last for an inter-stimuli interval duration in a range of about 2 seconds to about 30 seconds.

5. The illumination system of claim 1, wherein at least some stimulation sequence durations are in a range of about 30 seconds to about 5 minutes.

6. The illumination system of claim 1, wherein at least some inter-sequence intervals last for an inter-sequence interval duration on the order of 5 seconds to about 45 seconds.

7. The illumination system of claim 1, wherein the durations of at least some pulses and inter-pulse intervals are arranged to provide a light pulse duty cycle in a range of about 0.2-0.8.

8. The illumination system of claim 1, configured to provide at least some light pulses with an effective light level modulation depth of about 0.2 or more, and/or an effective light level modulation of about 50 lux or more.

9. The illumination system of claim 1, wherein at least some light pulses provide light with a limited spectrum in the range of about 700 to 600 nanometers (red to orange) or white light with a colour temperature of about 4000 K or more.

10. The illumination system of claim 1, further comprising at least one further light source providing a further illumination signal to the subject configured to influence the subjects' behaviour.

11. (canceled)

12. A method of operating an illumination system comprising a light source, the method comprising providing an illumination signal comprising
a plurality of light pulses having a pulse duration and being separated by inter-pulse intervals, where the light pulse durations are on the order of milliseconds to tens of milliseconds, the inter-pulse intervals last on the order of milliseconds to tens of milliseconds,
wherein the light pulses are grouped in stimuli which have a stimulus duration and are separated by inter-stimuli intervals, and
the stimuli are grouped in stimulation sequences which have a stimulation sequence duration and are separated by inter-sequence intervals,
wherein the stimulus duration is on the order of hundreds of milliseconds to tens of seconds, the inter-stimuli interval s last on the order of seconds to tens of seconds,
the stimulation sequence durations are on the order of tens of seconds to minutes, and the inter-sequence intervals last on the order of seconds to tens of seconds and longer than the inter-stimuli intervals.

13. The method of claim 12, further comprising illuminating a mammalian subject, in particular a human subject, with the illumination signal.

14. The method of claim 13, further comprising providing a further illumination signal to the subject configured to influence the subjects' behaviour.

15. A computer program product, implemented on a computer-readable storage medium, the computer program product configured for, when run on a computing device, executing the method according to claim 12.
